(19) United States
(12) Patent Application Publication
(10) Pub. No.: US 2010/0297206 A1
(43) Pub. Date: Nov. 25, 2010

(54) Antimicrobial Upconversion System

(75) Inventors: Jaehong Kim, Alpharetta, GA (US); Ezra Lucas Hoyt Cates, Atlanta, GA (US); Min Cho, Atlanta, GA (US)

Correspondence Address: TROUTMAN SANDERS LLP;5200 BANK OF AMERICA PLAZA, 600 PEACHTREE STREET, N.E., SUITE 5200, ATLANTA, GA, 30308-2216, US

(73) Assignee: Georgia Tech Research Corporation, Atlanta, GA (US)

(21) Appl. No.: 12/785,207

(22) Filed: May 21, 2010

Related U.S. Application Data
(60) Provisional application No. 61/180,165, filed on May 21, 2009.

(51) Int. Cl. A01N 25/08; A01N 59/16

(52) U.S. Cl. 424/409; 424/617

(57) ABSTRACT

Antimicrobial articles, systems, and methods may be used for killing, inactivating, and/or inhibiting microorganisms. The antimicrobial articles and systems utilize up-conversion luminescence wherein a phosphor or luminescent material is capable of absorbing visible, infrared light, or longer wavelength radiation and emitting antimicrobial ultraviolet radiation via upconversion thus inhibiting the growth of, inhibiting the reproduction of or killing or otherwise inactivating microorganisms such as, but not limited to, spores, bacteria, fungi, mildew, mold, and algae. Embodiments of the antimicrobial article or system may comprise such a luminescent material and thus will have antimicrobial activity when exposed to natural or artificial light.

### RELATED APPLICATIONS

This patent application claims priority under 35 U.S.C. §119 to U.S. Provisional Patent Application Ser. No. 61/180,165 file on May 21, 2009, which is hereby incorporated by reference in its entirety.

### FIELD OF THE INVENTION

The present invention is directed to antimicrobial articles, systems, and methods of using and making antimicrobial articles and systems. Embodiments of the antimicrobial articles and systems utilize up-conversion luminescence, wherein a phosphor or luminescent material is capable of absorbing visible, infrared light, or lower wavelength radiation and emitting antimicrobial ultraviolet radiation via upconversion.

### BACKGROUND

Ultraviolet (UV) light is electromagnetic radiation having a wavelength shorter than that of visible light and longer than x-rays and gamma rays. UV light is typically considered to include electromagnetic radiation having wavelengths in the range of from 10 nm to 400 nm. UV light is present to a certain degree in both sunlight and in artificial lights.

UV light has several applications. For example, UV light may be an ionizing radiation capable of providing energy to initiate certain chemical reactions and also has antimicrobial activity. The ultraviolet radiation is generally divided into three classifications, UV-A, UV-B, and UV-C. UV-A is typically considered to include electromagnetic radiation having wavelengths in the range from 400 nm-315 nm; UV-B includes electromagnetic radiation having wavelengths in the range 315 nm-280 nm; and UV-C includes electromagnetic radiation having wavelengths in the range 280 nm-100 nm.

The sun emits electromagnetic radiation including radiation in the IR, visible, and UV wavelength ranges. Further, the UV radiation from the sun includes the UVA, UVB, and UVC bands. However, the Earth's atmosphere blocks nearly all of the UV radiation from reaching the earth's surface, thus shielding organisms from the adverse effects on the living organisms.

Of the IR, visible and UV ranges, UV-C is considered to have the greatest antimicrobial activity. If a microorganism is exposed to UV-C radiation, for example, the UVC radiation disrupts and covalently binds complementary strands of DNA together thereby inhibiting transcription. Once the nucleic acids are disrupted, the microorganism loses its reproductive capabilities and the disruption of the nucleic acids ultimately results in the death of the microorganism. Thus, at certain wavelengths, UV-C radiation is mutagenic to bacteria, viruses and other microorganisms. Specifically, UV-C radiation having a wavelength of approximately 260 to approximately 265 nm is most effective for rendering the microorganism harmless to cause disease or illness and/or inhibiting further growth and reproduction.

The effectiveness of the antimicrobial activity of UV-C or other UV radiation depends on a number of factors including, but not limited to, the duration and intensity of the exposure to the radiation, the spectrum of the radiation, the efficiency of the exposure, and a specific microorganism's susceptibility to radiation. Increases in effectiveness and UV intensity can be achieved through reflection and/or concentration of the radiation.

Currently, solar water disinfection is used to disinfect water using only UV light from the sun. Though because only a portion of the solar spectrum that reaches the earth's surface consists of UV radiation, the process is largely inefficient. Other current antimicrobial materials rely on organic surface moieties which target the cell membrane to prevent the presence and growth of microorganisms on surfaces. However, these mechanisms rely on specific interactions, making many forms of microorganisms resistant, and rely on direct contact between the surface and the organism.

There exists a need for articles, systems and methods to expose potentially harmful microorganisms to UV radiation. The articles, systems and methods may provide “self sterilizing” articles.

### SUMMARY

Embodiments of the antimicrobial article for killing, inactivating, and/or inhibiting microorganisms comprise a luminescent material capable of absorbing visible or infrared light and emitting ultraviolet radiation via upconversion. The luminescent material comprises an activator capable of absorbing visible or infrared light and emitting radiation having a wavelength in the ultraviolet range via upconversion, a host material, and a sensitizer capable of absorbing visible or infrared light and transferring energy to the activator, without significantly emitting radiation having a wavelength in the ultraviolet range.

An additional embodiment of the antimicrobial article comprises a core of luminescent material capable of absorbing visible or infrared light and emitting ultraviolet radiation via upconversion, wherein the luminescent material comprises an activator capable of absorbing visible or infrared light and emitting ultraviolet radiation via upconversion and a host material and a coating of a transparent material encapsulating the core.

An embodiment of a system for the inhibition of microorganisms in water, comprises a water sterilizer comprising an interior, at least one wall, an inlet and an outlet, wherein substantially all visible and infrared light from outside the water sterilizer can pass into the interior of the water sterilizer. In certain embodiments, substantially no ultraviolet radiation from the interior of the water sterilizer can pass outside the water sterilizer. The sterilizer has a water source in communication with the inlet and a water supply in communication with the outlet. In addition, the system may comprise a solar concentrator for focusing visible light onto the water sterilizer. Embodiments of the system for inhibition of microorganisms in water also comprise an upconversion surface in the interior of the sterilizer, wherein the upconversion surface is capable of absorbing visible or infrared light entering the interior of the water sterilizer, and emitting ultraviolet radiation via upconversion, the ultraviolet radiation disinfecting or inhibiting growth of microorganisms in the interior of the water sterilizer.

Any of the embodiments of the antimicrobial article, systems or methods may comprise a sensitizer capable of absorbing visible or infrared light and transferring energy to the activator, without significantly emitting radiation having a wavelength in the ultraviolet range; a coactivator that is both capable of absorbing visible or infrared light and emitting ultraviolet radiation via upconversion and capable of absorbing visible or infrared light and transferring energy to the activator; metallic nanoparticles capable of producing surface plasmons; a photosensitizer capable of inactivating or inhibiting microorganisms, or combinations thereof.

Other aspects and features of embodiments of the present invention will become apparent to those of ordinary skill in the art, upon reviewing the following description of specific, exemplary embodiments of the present invention in concert with the figures. While features of the present invention may be discussed relative to certain embodiments and figures, all embodiments of the present invention can include one or more of the features discussed herein. While one or more embodiments may be discussed as having certain advantageous features, one or more of such features may also be used with the various embodiments of the invention discussed herein. In similar fashion, while exemplary embodiments may be discussed below as system or method embodiments it is to be understood that such exemplary embodiments can be implemented in various systems and methods.

### BRIEF DESCRIPTION OF THE FIGURES

FIG. 1 is the upconversion spectrum of a luminescent material comprising nanocrystalline (Pr_{0.012}Gd_{0.012}Li_{0.072}Y_{0.904})₂SiO₅ when exposed to radiation having a wavelength of 488 nm;

FIG. 2 shows the inactivation of Bacillus subtilis spores on a quartz cover slip coated with upconversion luminescent material (Pr_{0.01}Gd_{0.01}Y_{0.98})₂SiO₅ and placed under a fluorescent white light with a UV filter;

FIG. 3 depicts an embodiment of a system for the inhibition of microorganisms in water; the embodiment comprises a water sterilizer, a solar concentrator for focusing visible light onto the water sterilizer, and an upconversion surface in the interior of the sterilizer; and

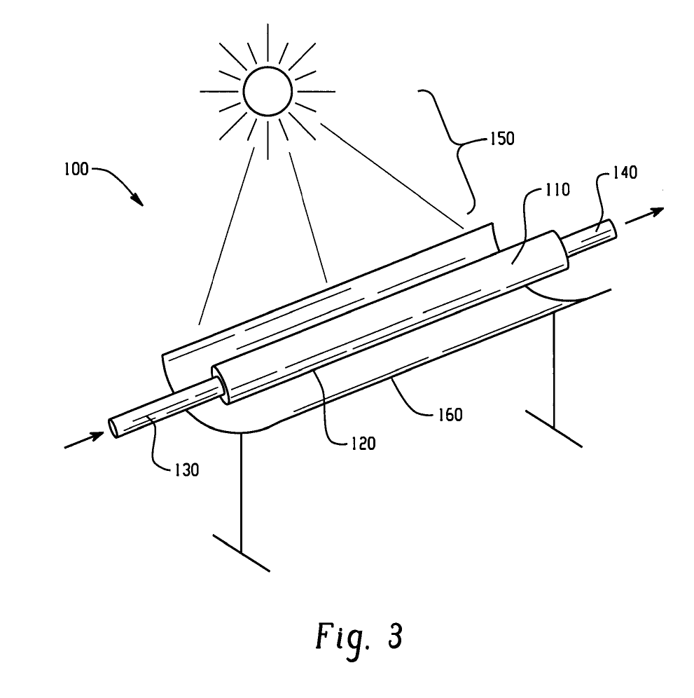

FIGS. 4A, 4B, and 4C are photographs that show the results of the antibacterial activity of upconversion luminescent materials under a fluorescent white light with a UV cut-off filter where FIG. 4A shows the biofilm on an uncoated surface after exposure to light; FIG. 4B shows the biofilm on a surface coated with a luminescent material without being exposed to visible light; and FIG. 5C shows the biofilm on a coated surface coated with luminescent material after exposure to visible light.

### DETAILED DESCRIPTION

The present invention is directed to antimicrobial articles, systems, and methods of using and making antimicrobial articles and systems. Embodiments of the antimicrobial articles and systems utilize up-conversion luminescence wherein a phosphor or luminescent material is capable of absorbing visible, infrared light, or longer wavelength radiation and emitting antimicrobial ultraviolet radiation via upconversion thus inhibiting the growth of, inhibiting the reproduction of or killing or otherwise inactivating microorganisms such as, but not limited to, spores, bacteria, fungi, mildew, mold, and algae. Embodiments of the antimicrobial article or system may comprise such a luminescent material and thus will have antimicrobial activity when exposed to natural or artificial light.

In specific embodiments, the luminescent material may comprise an activator capable of absorbing the visible or infrared light and emitting radiation having a wavelength in the ultraviolet range via upconversion. Visible light comprises the portion of the electromagnetic spectrum that is visible to or can be detected by the human eye. Visible light is typically considered to include electromagnetic radiation having wavelengths in the range of about 390 to 750 nm.

Infrared radiation (IR) includes electromagnetic radiation having a wavelength in the range of 700 to 3000 nm. IR radiation has a wavelength that is longer and a frequency that is lower than that of visible light.

Certain chemical compounds have the ability to absorb radiation with longer wavelengths and emit radiation with a shorter wavelength and higher energy. These compounds are considered to be up-converting materials. As used herein, “up conversion” means absorbing radiation with longer wavelengths and emitting radiation with shorter wavelengths and higher energy. The term up-converting is used because the absorbed longer wavelength radiation has lower energy than the shorter wavelength radiation. The luminescent materials must absorb a few photons of lower energy to emit the higher energy radiation. In certain embodiments, light incident on an upconverting material may be absorbed by the material in the infrared and visible ranges and up-converted such that the upconverting material emits ultraviolet radiation, for example. In such an embodiment, a portion of the spectrum of the solar radiation at the earth's surface may be converted to an antimicrobial UV radiation.

As used herein, “at least one”, “a”, or “an” means one or more and includes individual components as well as mixtures or combinations of the individual components.

“Antimicrobial” or “Biocidal” refer to the ability to inhibit the growth of, inhibit the reproduction of or kill or inactivate microorganisms such as, but not limited to, spores, bacteria, fungi, mildew, mold, viruses, protozoa, and algae.

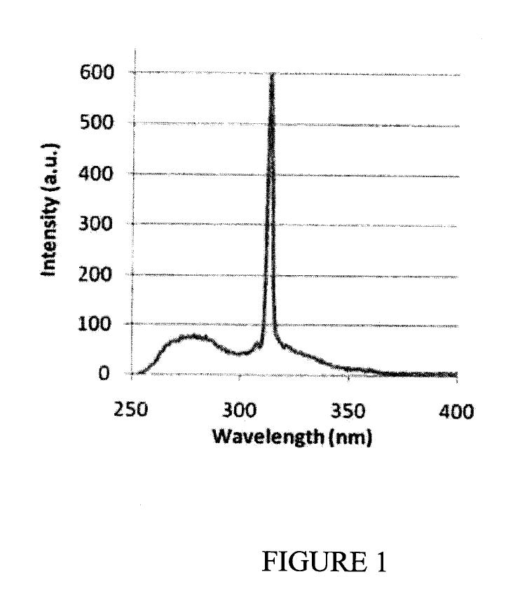

Embodiments of the antimicrobial articles include antimicrobial articles and methods for killing, inactivating, and/or inhibiting of microorganisms. In one embodiment, the antimicrobial may comprise an upconverting luminescent material. As used herein, a “luminescent material” is any substance or phosphor that is capable of absorbing photons that would result in radiation of photons from the substance. Up-converting luminescent materials are capable of absorbing multiple photons of lower energy and emitting a photon of higher energy. For example, as shown in FIG. 1, when exposed to radiation having a wavelength of 488 nm, a luminescent material comprising nanocrystalline (Pr_{0.012}Gd_{0.012}Li_{0.072}Y_{H0.904})₂SiO₅ emits a radiation spectrum having a shallow broad peak in the antimicrobial UVC range and a sharp, intense peak at 313 nm. The emission peak was determined by laser induced photoluminescence spectroscopy of the pressed powder sample.

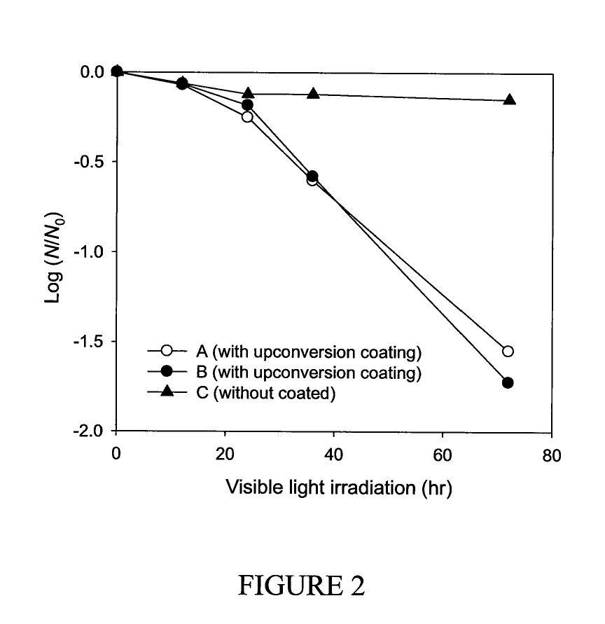

Up-converting luminescent materials may be incorporated into various articles. The up-converting luminescent materials may be incorporated into or onto the antimicrobial articles by any method including, but not limited to, by spraying, dipping, or painting onto the article, by incorporating the luminescent material in the injection molding or extrusion process, impregnation into fibers or fabrics, for example. If the article is then exposed to visible or infrared light, the article will emit antimicrobial UV radiation such as, but not limited to, UV-C radiation. Embodiments of the luminescent material may comprise a host material and an activator capable of absorbing visible or infrared light and emitting radiation having a wavelength in the ultraviolet range via upconversion. As shown in FIG. 2, a upconverting luminescent material comprising (Pr_{0.01}Gd_{0.01}Y_{0.98})SiO₅ and placed under a fluorescent white light with a UV filter results in the inactivation of Bacillus subtilis spores on a quartz cover slip.

The luminescent material and/or the antimicrobial article may further comprise a sensitizer capable of absorbing visible or infrared light and transferring energy to the activator, without significantly emitting radiation having a wavelength in the ultraviolet range. A sensitizer may increase the efficiency of the antimicrobial article by having a different absorption spectrum as the activator or by increasing the efficiency of the absorption of the incident radiation in the same range as the activator.

The luminescent material and/or the antimicrobial article may further comprise a coactivator that is both capable of absorbing visible or infrared light and emitting ultraviolet radiation via upconversion and capable of absorbing visible or infrared light and transferring energy to the activator. Similarly to the sensitizer, the coactivator may increase the efficiency of the antimicrobial article by having a different absorption spectrum as the activator or by increasing the efficiency of the absorption of the incident radiation in the same range as the activator. Further, the emission spectrum of the coactivator may complement the emission spectrum of the activator and result in more effective antimicrobial activity with regard to a wider variety of microorganisms.

The luminescent material and/or the antimicrobial article may further comprise metallic nanoparticles capable of producing surface plasmons. The surface plasmons may be excited by light to produce a localized surface plasmon resonance which can transfer energy to activator centers.

Embodiments of the luminescent material and the antimicrobial article comprise a host material. The bulk crystal may be doped with the other components that provide the up-converting antimicrobial properties to the material.

Generally, host materials comprise oxides, halides, sulfides, and selenides. Suitable host materials may comprise at least one oxide, halide, sulfide, or selenide of at least one of gadolinium, yttrium, lanthanum, lutetium, sodium, lithium, potassium, barium, strontium, calcium, magnesium, and combinations of these materials. Host materials include, but are not limited to, transition metal oxides or transition metal halides of a crystalline, nano-crystalline, micro-crystalline, polycrystalline or amorphous form. Embodiments of the host material may be, for example, transition metal oxides, transition metal halides or combinations of transition metal oxides and transition metal halides of a crystalline, nano-crystalline, micro-crystalline, polycrystalline or amorphous form. Particular embodiments of such crystal matrices which may comprise the host material include oxy-sulfides, oxy-fluorides, oxy-chlorides, or vanadates of these various metals.

Embodiments of the host materials include, but are not limited to, sodium yttrium fluoride (NaYF₄), lanthanum fluoride (LaF₃), lanthanum oxysulfide (La₂O₂S), yttrium oxysulfide (Y₂O₂S), yttrium fluoride (YF₃), yttrium gallate, yttrium aluminum garnet (YAG), gadolinium fluoride (GdF₃), barium yttrium fluoride (BaYF₅, BaY₂F₈), gadolinium oxysulfide (Gd₂O₂S), calcium tungstate (CaWO₄), yttrium oxide (Y₂O₃); and lanthanam oxysulphide (La₂O₂S).

Additional host materials include NaGdF₄, LiGdF₄, KGdF₄, BaGd₂F₈, SrGd₂F₈, CaGd₂F₈, MgGd₂F₈, NaLaF₄, LiLaF₄, KLaF₄, NaLuF₄, LiLuF₄, KLuF₄, LuF₃, LiYF₄, KYF₄, BaKa₂F₈, SrKa₂F₈, CaKa₂F₈, MgKa₂F₈, BaLu₂F₈, SrLu₂F₈, CaLu₂F₈, or MgLu₂F₈, BaY₂F₈, CaY₂F₈, MgY₂F₈, and combinations thereof.

In further embodiments, the antimicrobial article or the luminescent material comprises a host comprising a halide. The inventors have found that hosts comprising halides are more efficient than other hosts materials for use in upconverting IR and visible light for antimicrobial articles. Specific embodiments of the host materials that have particular applicability for the luminescent materials for antimicrobial articles include NaGdF₄, NaLaF₄, LuF₃, NaLuF₄, BaLa₂F₈, BaGd₂F₈, and BaLu₂F₈. Further, in any host material containing sodium, the Na⁺ may partially or completely substituted with either lithium or potassium. Additionally, in any host material containing barium, the Ba²⁺ may also be partially or completely substituted with Sr²⁺, Ca²⁺, or Mg²⁺.

Embodiments of the luminescent material comprise an activator or phosphor (hereinafter “activator”). Activators may include any optically active ion that may be doped into a host material and is capable of absorbing visible, infrared light or lower energy radiation and emitting radiation having a wavelength in the ultraviolet range via upconversion. Activators may emit a spectrum of radiation wherein at least a portion of the radiation has a wavelength in the range from 150 nm to 400 nm.

Embodiments of activators that may be doped into the host material include, but are not limited to, include including cerium (Ce), praseodymium (Pr), neodymium (Nd), samarium (Sm), europium (Eu), gadolinium (Gd), terbium (Tb), dysprosium (Dy), holmium (Ho), erbium (Er), thulium (Tm), and combinations of these materials. In additional embodiments, the activator is praseodymium, erbium, thulium, holmium, ytterbium, gadolinium or combinations of these elements; further activators are Pr³⁺, Tm³⁺, Er³⁺, Gd³⁺ and combinations of Pr³⁺, Tm³⁺, Er³⁺, Gd³⁺. In a specific embodiment of the luminescent material comprises a host material comprising Gd³⁺ and the activator also comprises Gd³⁺.

The organic and/or inorganic activator or phosphors may be present in the disclosed composition in a doping percentage ranging from 0.01% to 60%. As used herein, the “doping percentage” is the number percent of host cations that are substituted with dopant, also referred to as “atomic percent.”

The luminescent materials typically are incorporated into antimicrobial articles with an average particle size ranging from 1 nm to 1 cm, for example.

Embodiments of the antimicrobial articles and the luminescent materials may further comprise a sensitizer. A sensitizers is a dopant that is capable of absorbing incident light and transfer energy from the incident light to the activator, however, sensitizers do not emit electromagnetic radiation in any target wavelength themselves. A sensitizer may complement and increase the efficiency of the activator if it has a different absorption spectrum than the activator, for example. Sensitizers include, but are not limited to, ytterbium, praseodymium, and thulium.

Embodiments of the antimicrobial articles and the luminescent materials may further comprise a coactivator. A coactivator is any dopant that may added to a host material that is capable of acting as a sensitizer and also emit, at least to some extent, radiation in the desired wavelengths. A coactivator may complement and increase the efficiency of the activator if it has a different absorption spectrum and/or a different emission spectrum than the activator, for example. Coactivators include, but are not limited to, Pr, Ho, Tb, Tm, Er or a combination of any of Pr, Ho, Tb, Tm, or Er.

Embodiments of the antimicrobial articles and the luminescent materials may further comprise metallic nanoparticles. The metallic nanoparticles may comprise, for example, copper, silver, gold or combinations of at least two of copper, silver, and gold. Utilizing upconversion materials in conjunction with Cu, Ag, or Au nanoparticles incorporated into an amorphous material improves upconversion through surface plasmon resonance. Localized surface plasmon resonance results in electron charge oscillation in the metallic nanoparticles when excited by light. The electron charge oscillation may transfer energy from the metallic nanoparticle to the activator, coactivator, and/or the sensitizer to increase the efficiency and emission output of the luminescent material and/or the antimicrobial article.

Further embodiments of an antimicrobial article may comprise a luminescent material capable of absorbing visible or infrared light and emitting ultraviolet radiation via upconversion and a photosensitizer. A photosensitizer is light activated antimicrobial agent capable of inactivating or inhibiting microorganisms through a different mechanism than emission of UV radiation. However, the antimicrobial activity and/or the efficiency of the photosensitizer is increased by the ultraviolet radiation emitted from the luminescent material. As in other embodiments, the antimicrobial article and luminescent material in this embodiment may comprise an activator capable of absorbing visible or infrared light and emitting radiation having a wavelength in the ultraviolet range via upconversion, a host material, and/or a sensitizer capable of absorbing visible or infrared light and transferring energy to the activator, without significantly emitting radiation having a wavelength in the ultraviolet range.

The photosensitizers include any material which catalyzes a chemical reaction upon irradiation, for example, the photosensitizer may be a photocatalyst, organic compound containing porphyrin moieties, organometallic complex, a semiconductor oxide or any combination thereof, which is capable of generating reactive oxygen species. Photosensitizers include, but are not limited to, titanium dioxide, zinc oxides, fullerenes, and organic compounds containing porphyrin moieties, organometallic complexes, or combinations thereof.

### Embodiments/Combinations

The following table illustrates by way of example some embodiments of the luminescent material that may be incorporated into a luminescent material.

TABLE 1
Embodiments of luminescent materials

| | | Coactivators/Sensitizers |
|---|---|---|
| | | (alone or in any |
| Host | Activator | combination) |
| NaGdF₄ | Gd | Pr, Yb, Ho, Tb, Tm, Er |
| LiGdF₄ | Gd | Pr, Yb, Ho, Tb, Tm, Er |
| KGdF₄ | Gd | Pr, Yb, Ho, Tb, Tm, Er |
| GdF₃ | Gd | Ho, Tb, Tm, Er |
| NaLaF₄ | Gd, Pr, Tm, or, Er | Pr, Yb, Ho, Tb, Tm, Er |
| LiLaF₄ | Gd, Pr, Tm, or, Er | Pr, Yb, Ho, Tb, Tm, Er |
| KLaF₄ | Gd, Pr, Tm, or, Er | Pr, Yb, Ho, Tb, Tm, Er |
| NaLuF₄ | Gd, Pr, Tm, or, Er | Pr, Yb, Ho, Tb, Tm, Er |
| LiLuF₄ | Gd, Pr, Tm, or, Er | Pr, Yb, Ho, Tb, Tm, Er |
| KLuF₄ | Gd, Pr, Tm, or, Er | Pr, Yb, Ho, Tb, Tm, Er |
| LuF₃ | Gd, Pr, Tm, or, Er | Pr, Yb, Ho, Tb, Tm, Er |
| NaYF₄ | Gd | Pr, Yb, Ho, Tb, Tm, Er |
| NaYF₄ | Tm or Er | Pr, Ho, Tb, Tm, Er |
| LiYF₄ | Gd, Pr, Tm, or, Er | Pr, Yb, Ho, Tb, Tm, Er |
| KYF₄ | Gd, Pr, Tm, or, Er | Pr, Yb, Ho, Tb, Tm, Er |
| BaY₂F₈ | Gd, Tm, or, Er | Pr, Ho, Tb, Tm, Er |
| SrY₂F₈ | Pr, Er, or Tm | Pr, Yb, Ho, Tb, Tm, Er |
| CaY₂F₈ | Pr, Er, or Tm | Pr, Yb, Ho, Tb, Tm, Er |
| MgY₂F₈ | Pr, Er, or Tm | Pr, Yb, Ho, Tb, Tm, Er |
| BaGd₂F₈ | Gd | Pr, Yb, Ho, Tb, Tm, Er |
| SrGd₂F₈ | Gd | Pr, Yb, Ho, Tb, Tm, Er |
| CaGd₂F₈ | Gd | Pr, Yb, Ho, Tb, Tm, Er |
| MgGd₂F₈ | Gd | Pr, Yb, Ho, Tb, Tm, Er |
| BaLa₂F₈ | Gd, Pr, Er, or Tm | Pr, Yb, Ho, Tb, Tm, Er |
| SrLa₂F₈ | Gd, Pr, Er, or Tm | Pr, Yb, Ho, Tb, Tm, Er |
| CaLa₂F₈ | Gd, Pr, Er, or Tm | Pr, Yb, Ho, Tb, Tm, Er |
| MgLa₂F₈ | Gd, Pr, Er, or Tm | Pr, Yb, Ho, Tb, Tm, Er |
| BaLu₂F₈ | Gd, Pr, Er, or Tm | Pr, Yb, Ho, Tb, Tm, Er |
| SrLu₂F₈ | Gd, Pr, Er, or Tm | Pr, Yb, Ho, Tb, Tm, Er |
| CaLu₂F₈ | Gd, Pr, Er, or Tm | Pr, Yb, Ho, Tb, Tm, Er |
| MgLu₂F₈ | Gd, Pr, Er, or Tm | Pr, Yb, Ho, Tb, Tm, Er |

Further embodiments of the invention include an antimicrobial article for killing, inactivating, and/or inhibiting of microorganisms, comprising a core of luminescent material capable of absorbing visible or infrared light and emitting ultraviolet radiation via upconversion and a coating of a transparent material encapsulating the core. The coating may further comprise a sensitizer. As in other embodiments, the luminescent material may comprise an activator capable of absorbing visible or infrared light and emitting ultraviolet radiation via upconversion and a host material.

The coating may be any material that is transparent or semitransparent to radiation in the absorption and emission spectrum of the activator, sensitizer, and/or the coactivator and may partially, substantially or completely encapsulate the luminescent material. The coating may comprise the same material as the host, a polymer or silicon dioxide, for example. In further embodiments, the coating may comprise an anti-reflective coating to decrease the loss of incident infrared or visible radiation.

To further enhance the antimicrobial activity, the coating may further comprise components that increase the efficiency of the activator in the core. For example, the coating material may be doped with a coactivator, sensitizer, metallic nanoparticles, or photosensitizers, for example. Coating phosphor crystal particles with an undoped or partially doped layer may significantly enhance the optical efficiency of the luminescent material.

Embodiments of the antimicrobial articles, luminescent materials, and systems may be used for many applications where control of microorganisms is desired. Specifically, embodiments of the antimicrobial articles, luminescent materials, and systems may be used in applications wherein chemical sterilization is not desired or practical. Embodiments of the antimicrobial articles and surfaces that may include up-converting luminescent materials include, but are not limited to, water sterilization, interior materials of buildings, automobiles, aircraft and/or ships, food packaging, fluid containers, water containers, everyday household items such as countertops, dishwashers, cabinets, appliance surfaces, tableware, food storage containers, sinks, tubs, toilets, urinals, bidets, bath enclosures, wallpaper, paints, grout, caulk, tiles, contact paper, wipes and towels, plastic pools, pool liners, toys, pipes, wood, flooring (especially bathroom and locker room floor), trash receptacles, and glass; personal care items, such as, toothbrushes, razors, nippers, scissors, grooming devices, clothing, and shoe liners, and sinks, as well as military applications, such as anti-biological weapons equipment, clothing, masks, glasses, helmets, air filters and respirators; laboratory equipment, such as glassware, utensils, and countertops; medical equipments such as surgical instruments, laparoscopic or arthroscopic instruments, catheters, sutures, wound dressings, bandages, compresses, packaging materials, wipes, and towels; food service equipment, such as, countertops, appliance surfaces, tableware, food storage containers, commercial food packaging, such as bags, wrappers, liners, wipes and towels; industrial applications, such as, components of cooling towers, filters, clean room filtration equipment, air conditioning units, and food processing equipment, such as, chicken and seafood processing equipment; and other items, such as, boat hulls, aquarium surfaces, papers, fibers, fabrics, glass, ceramics, metals, and plastics.

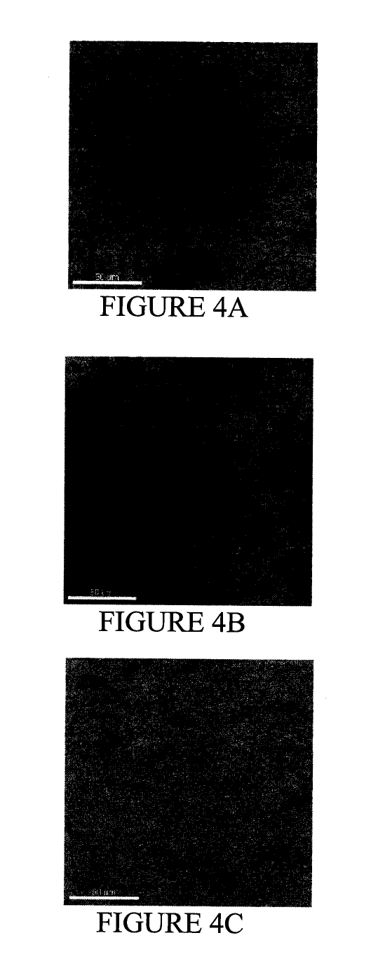

Embodiments of the antimicrobial articles, luminescent materials, systems, and methods may be used for the inhibition of microorganisms in water. An embodiment of a water sterilization system 100 is shown in FIG. 4. Such embodiments may comprise a water sterilizer 110 comprising an interior, at least one wall 120, an inlet 130 and an outlet 140, wherein visible and/or infrared light is present in the water sterilizer from visible and/or infrared light 150 passing from outside the water sterilizer into the interior of the water sterilizer. In another embodiment, the water sterilizer may comprise interior lighting, such as artificial incandescent or fluorescent lighting. The visible and/or infrared light may be upconverted to ultraviolet light by a luminescent material capable of absorbing visible or infrared light and emitting ultraviolet radiation. In a further embodiment, the sterilizer 110 may comprise a wall material, wherein the wall material that allows substantially no ultraviolet radiation from the interior of the water sterilizer to pass outside the water sterilizer. The water sterilizer 110 may further comprise a water source in communication with the inlet 130 and a water supply in communication with the outlet 140. Water contaminated with microorganisms may be fed into the water sterilizer through the water inlet 130. Once with the interior of the water sterilizer 110, the visible and/or infrared light may be upconverted to radiation in the ultraviolet range to kill, inactivate, and/or inhibit the microorganisms in the water. Thus, the water exiting the water sterilizer through the outlet 140 is now potable water.

Other designs include a flat reactor with laminar flow, in which case the backing is coated with the material, and may also include reflective backings behind the material, and an anti-reflective coating on top of the material, similar to solar cell technology.

Embodiments of the system may further comprise a solar concentrator 160 for focusing visible light onto or into the water sterilizer 110. The higher the intensity of light capable of being absorbed that is incident upon the luminescent material will directly result in a greater the intensity of the emitted ultraviolet light. The luminescent material may be present on an upconversion surface in the interior of the sterilizer 110, wherein the upconversion surface is capable of absorbing visible or infrared light entering the interior of the water sterilizer, and emitting ultraviolet radiation via upconversion, the ultraviolet radiation disinfecting or inhibiting growth of microorganisms in the interior of the water sterilizer 110.

An additional embodiment of an antimicrobial fluid container for killing, inactivating, and/or inhibiting of microorganisms may comprise a container comprising transparent portions, wherein the transparent portions comprise particles of luminescent material capable of absorbing visible or infrared light and emitting ultraviolet radiation via upconversion. As in other embodiments, the luminescent material comprises an activator capable of absorbing visible or infrared light and emitting ultraviolet radiation via upconversion.

The transparent material of the container may further comprise components that increase the efficiency or performance of the activator in the core. For example, the transparent material may be doped with a coactivator, sensitizer, metallic nanoparticles, or photosensitizers, for example.

### EXAMPLES

### Example 1

### Microbial Inactivation of Bacillus subtilis

Microbial inactivation experiments were conducted using Bacillus subtilis spores (ATCC 6633), cultured in 1/10 nutrient agar with an extended incubation time of 6 days and counted with spreading plate method. Three samples were prepared, Samples A and B comprised a coating of an upconverting material, (Pr_{0.01}Gd_{0.01}Y_{0.98})SiO₅ on a quartz slip, and Sample C was uncoated. Bacillus subtilis spores were applied to each sample. All three samples were placed under a fluorescent white light with a UV filter. As shown in FIG. 2, the upconverting luminescent material, (Pr_{0.01}Gd_{0.01}Y_{0.98})SiO₅, coated on Samples A and B resulted in ˜1.5 log(˜97%) inactivation of the Bacillus subtilis after 72 hr. (−1 and −2 log on Y axis indicates the 90% and 99% inactivation of microorganisms, respectively.) In contrast, there was no significant inactivation observed on Sample C without upconverting material.

In summary, an upconverting luminescent material comprising (Pr_{0.01}Gd_{0.01}Y_{0.98})SiO₅ and placed under a fluorescent white light with a UV filter results in the inactivation of Bacillus subtilis spores.

### Example 2

### Microbial Inactivation of Pseudomonas aeruginosa

Pseudomonas aeruginosa (PA01) biofilm was developed with Center for Disease Control biofilm reactor and was quantitatively assayed with scanning confocal laser micrographs after fluorescence dye staining. The biofilms were exposed to fluorescent light with a UV cut-off filter. In the micrographs shown in FIGS. 4A, 4B, and 4C, green color indicates live PA1 and red color indicates dead PA01. FIG. 4C shows the micrograph of an upconverting luminescent material coated surface exhibits biofilm growth inhibition, which indicates the antibacterial activity of upconversion materials. In contrast, the biofilm on the surface coated with a luminescent material without being exposed to visible light shown in FIG. 4B and the biofilm on the coated surface coated with luminescent material after exposure to visible light shown in FIG. 4C clearly show the bacterial growth and biofilm formation of PA01.

The embodiments of the described method and systems are not limited to the particular formulations, method steps, and materials disclosed herein as such formulations, process steps, and materials may vary somewhat. Moreover, the terminology employed herein is used for the purpose of describing exemplary embodiments only and the terminology is not intended to be limiting since the scope of the various embodiments of the present invention will be limited only by the appended claims and equivalents thereof.

Therefore, while embodiments of the invention are described with reference to exemplary embodiments, those skilled in the art will understand that variations and modifications can be effected within the scope of the invention as defined in the appended claims. Accordingly, the scope of the various embodiments of the present invention should not be limited to the above discussed embodiments, and should only be defined by the following claims and all equivalents.

1. An antimicrobial article for killing, inactivating, and/or inhibiting microorganisms, comprising:
a luminescent material capable of absorbing visible or infrared light and emitting ultraviolet radiation via upconversion, wherein the luminescent material comprises:
an activator capable of absorbing visible or infrared light and emitting radiation having a wavelength in the ultraviolet range via upconversion;
a host material; and
a sensitizer capable of absorbing visible or infrared light and transferring energy to the activator, without significantly emitting radiation having a wavelength in the ultraviolet range.

2. The antimicrobial article of claim 1, wherein the luminescent material further comprises metallic nanoparticles of copper, silver or gold.

3. The antimicrobial article of claim 1, wherein the luminescent material further comprises a coactivator that is both capable of absorbing visible or infrared light and emitting ultraviolet radiation via upconversion and capable of absorbing visible or infrared light and transferring energy to the activator.

4. The antimicrobial article of claim 1, wherein the host material comprises a halide.

5. The antimicrobial article of claim 1, wherein the activator is selected from the group consisting of Pr³⁺, Tm³⁺, Er³⁺, and Gd³⁺.

6. The antimicrobial article of claim 1, wherein the host material comprises Gd³⁺ and the activator is Gd³⁺.

7. The antimicrobial article of claim 1, wherein the activator is Tm³⁺.

8. The antimicrobial article of claim 1, wherein the activator is Er³⁺.

9. The antimicrobial article of claim 1, further comprising a coating encapsulating the luminescent material.

10. The antimicrobial article of claim 9, wherein the coating is doped with a coactivator that is both capable of absorbing visible or infrared light and emitting ultraviolet radiation via upconversion and capable of absorbing visible or infrared light and transferring energy to the activator.

11. The antimicrobial article of claim 1, wherein the activator is Gd; the host material is selected from the group consisting of NaGdF₄, LiGdF₄, KGdF₄, GdF₃, NaYF₄, BaGd₂F₈, SrGd₂F₈, CaGd₂F₈, MgGd₂F₈; and the sensitizer is Pr, Yb, Ho, Tm, Er or a combination of any of Pr, Yb, Ho, Tm, or Er.

12. The antimicrobial article of claim 1, wherein the activator is Gd, Pr, Tm, or Er; the host material is selected from the group consisting of NaLaF₄, LiLaF₄, NaLuF₄, LiLuF₄, KLuF₄, LuF₃, LiYF₄, KYF₄, BaKa₂F₈, SrKa₂F₈, CaKa₂F₈, MgKa₂F₈, BaLu₂F₈, SrLu₂F₈, CaLu₂F₈, or MgLu₂F₈; and the sensitizer is Pr, Yb, Ho, Tm, Er or a combination of any of Pr, Yb, Ho, Tm, or Er.

13. The antimicrobial article of claim 1, wherein the activator is Tm, or Er; the host material is NaYF₄; and the sensitizer is Pr, Ho, Tm, Er or a combination of any of Pr, Yb, Ho, Tm, or Er.

14. The antimicrobial article of claim 1, wherein the activator is Gd, Tm, or Er; the host material is BaY₂F₈; and the sensitizer is Pr, Ho, Tb, Tm, Er or a combination of any of Pr, Ho, Tb, Tm, or Er.

15. The antimicrobial article of claim 1, wherein the activator is Pr, Er, or Tm; the host material is selected from the group consisting of BaY₂F₈, CaY₂F₈ and MgY₂F₈; and the sensitizer is Pr, Yb, Ho, Tb, Tm, Er or a combination of any of Pr, Yb, Ho, Tb, Tm, or Er.

16. The antimicrobial article of claim 1, wherein the luminescent material is in a coating on the surface of the article.

17. The antimicrobial article of claim 1, wherein activators emit radiation with a wavelength in the range from 150 nm to 400 nm.

18. The antimicrobial article of claim 1, wherein the luminescent materials comprise transition metal oxides, transition metal halides or combinations of transition metal oxides and transition metal halides of a crystalline, nano-crystalline, micro-crystalline, polycrystalline or amorphous form which is doped with one or more rare-earth ions selected from the group consisting of Ce, Pr, Nd, Sm, Eu, Gd, Tb, Dy, Ho, Er, Tm, and Yb.

19. The antimicrobial article of claim 9, wherein the coating is an anti-reflective coating to decrease the loss of incident infra-red or visible radiation.

20. An antimicrobial article for killing, inactivating, and/or inhibiting microorganisms, comprising:
a core of luminescent material capable of absorbing visible or infrared light and emitting ultraviolet radiation via upconversion, wherein the luminescent material comprises:
an activator capable of absorbing visible or infrared light and emitting ultraviolet radiation via upconversion; and
a host material; and
a coating of a transparent material encapsulating the core.

21. The antimicrobial article of claim 20, wherein the coating comprises a sensitizer capable of absorbing visible or infrared light and transferring energy to the activator, without significantly emitting electromagnetic radiation having a wavelength in the ultraviolet range.

22. The antimicrobial article of claim 20, wherein the coating comprises the same material as the host material.

23. The antimicrobial article of claim 20, wherein the coating comprises silicon dioxide.

24. The antimicrobial article of claim 20, wherein the luminescent material comprises metallic nanoparticles of copper, silver or gold.

25. A system for the inhibition of microorganisms in water, comprising:
a water sterilizer comprising an interior, at least one wall, an inlet and an outlet, wherein substantially all visible and infrared light from outside the water sterilizer can pass into the interior of the water sterilizer, and wherein substantially no ultraviolet radiation from the interior of the water sterilizer can pass outside the water sterilizer;
a water source in communication with the inlet, and a water supply in communication with the outlet;
a solar concentrator for focusing visible light onto the water sterilizer; and
an upconversion surface in the interior of the sterilizer, wherein the upconversion surface is capable of absorbing visible or infrared light entering the interior of the water sterilizer, and emitting ultraviolet radiation via upconversion, the ultraviolet radiation disinfecting or inhibiting growth of microorganisms in the interior of the water sterilizer.

26. An antimicrobial fluid container for killing, inactivating, and/or inhibiting of microorganisms, comprising:
a container comprising transparent portions, wherein the transparent portions comprise particles of luminescent material capable of absorbing visible or infrared light and emitting ultraviolet radiation via upconversion, wherein the luminescent material comprises:
an activator capable of absorbing visible or infrared light and emitting ultraviolet radiation via upconversion; and
a host material.

27. The antimicrobial fluid container of claim 26, wherein the ultraviolet radiation includes radiation in the UVC range.

28. The antimicrobial fluid container of claim 26, wherein the transparent portion comprises a sensitizer capable of absorbing visible or infrared light and transferring energy to the activator, without significantly emitting electromagnetic radiation having a wavelength in the ultraviolet range.

29. The antimicrobial fluid container of claim 26, wherein the coating comprises the same material as the host material.

30. The antimicrobial fluid container of claim 26, wherein the coating comprises silicon dioxide.

31. The antimicrobial fluid container of claim 26, wherein the luminescent material comprises nanoparticles of copper, silver or gold.

32. An antimicrobial article, comprising:
a luminescent material capable of absorbing visible or infrared light and emitting ultraviolet radiation via upconversion, wherein the luminescent material comprises:
an activator capable of absorbing visible or infrared light and emitting radiation having a wavelength in the ultraviolet range via upconversion;
a host material; and
a sensitizer capable of absorbing visible or infrared light and transferring energy to the activator, without significantly emitting radiation having a wavelength in the ultraviolet range; and
a photo sensitizer, wherein the photosensitizer is capable of inactivating or inhibiting microorganisms and the performance of the photosensitizer benefits from ultraviolet radiation emitted from the luminescent material.

33. The antimicrobial article of claim 32, wherein the photo sensitizers include any material which catalyzes a chemical reaction upon irradiation.

34. The antimicrobial article of claim 32, wherein the photosensitizer is a photocatalyst, organic compound containing porphyrin moieties, organometallic complex, or any combination thereof, which is capable of generating reactive oxygen species.

35. The antimicrobial article of claim 34, wherein the photocatalyst is a semiconductor oxide.

36. The antimicrobial article of claim 35, wherein the semiconductor oxide is selected from the group consisting of titanium dioxide, zinc oxides, organic compounds containing porphyrin moieties, organometallic complexes or combinations thereof.

37. A method of killing, inactivating, and/or inhibiting microorganisms, comprising:
exposing the microorganisms to an antimicrobial article comprising a luminescent material capable of absorbing visible or infrared light and emitting ultraviolet radiation via upconversion, wherein the luminescent material comprises:
an activator capable of absorbing visible or infrared light and emitting radiation having a wavelength in the ultraviolet range via upconversion a host material; and
a sensitizer capable of absorbing visible or infrared light and transferring energy to the activator, without significantly emitting radiation having a wavelength in the ultraviolet range.

38. The method of claim 37, wherein the luminescent material further comprises metallic nanoparticles of copper, silver or gold.

39. The method of claim 37, wherein the luminescent material further comprises a coactivator that is both capable of absorbing visible or infrared light and emitting ultraviolet radiation via upconversion and capable of absorbing visible or infrared light and transferring energy to the activator.

40. The method of claim 37, wherein the host material comprises a halide.

41. The method of claim 37, wherein the activator is selected from the group consisting of Pr³⁺, Tm³⁺, Er³⁺, and Gd³⁺.

42. The method of claim 37, further comprising a coating encapsulating the luminescent material.

43. The method of claim 42, wherein the coating is doped with a coactivator that is both capable of absorbing visible or infrared light and emitting ultraviolet radiation via upconversion and capable of absorbing visible or infrared light and transferring energy to the activator.

44. The method of claim 37, wherein activators emit radiation with a wavelength in the range from 150 nm to 400 nm.

45. The method of claim 37, wherein the luminescent materials comprise transition metal oxides, transition metal halides or combinations of transition metal oxides and transition metal halides of a crystalline, nano-crystalline, micro-crystalline, polycrystalline or amorphous form which is doped with one or more rare-earth ions selected from the group consisting of Ce, Pr, Nd, Sm, Eu, Gd, Tb, Dy, Ho, Er, Tm, and Yb.

46. The method of claim 42, wherein the coating is an anti-reflective coating to decrease the loss of incident infra-red or visible radiation.

47. The method of claim 37, wherein the microorganism are at least one microorganism from the group comprising spores, bacteria, fungi, mildew, mold, viruses, protozoa, and algae.
